(19) United States
(12) Patent Application Publication
(10) Pub. No.: US 2013/0204741 A1
(43) Pub. Date: Aug. 8, 2013

(54) NEEDLE-LESS/WIRELESS ACUPUNCTURE SYSTEM AND METHOD

(71) Applicant: WENDELL MARTIN UNDERWOOD, TAMPA, FL (US)

(72) Inventor: WENDELL MARTIN UNDERWOOD, TAMPA, FL (US)

(21) Appl. No.: 13/427,000

(22) Filed: Mar. 22, 2012

Related U.S. Application Data
(60) Provisional application No. 61/594,045, filed on Feb. 2, 2012.

(51) Int. Cl. A61N 1/36

(52) U.S. Cl. CPC A61N 1/36003; G06Q 30/0633
USPC 705/26.8; 607/48

(57) ABSTRACT

A bio-electricity system and method utilizes electrical muscle stimulation, interferential current, micro-current, Russian stimulation and tens-transcutaneous electric nerve stimulation. The system and method are adapted for treating maladies of physically out-of-balance users according to the teachings of an ancient art. Such treatment is for pain relief, reduction of inflammation, decreasing muscle spasms and reduction of edema to other areas outside of the acupoint area. The system and method include a plurality of components including probes, adhesive, electronic components, an instruction manual, an electronic device and a program in the electronic device to wirelessly activate probes positioned on a user in accordance with the instruction manual.

### RELATED APPLICATION

The present application is based upon Provisional Application No. 61/594,045 filed Feb. 2, 2012, the subject matter of which is incorporated herein by reference.

### BACKGROUND OF THE INVENTION

1. Field of the Invention

The present invention relates to a needle-less/wireless acupuncture system and method and more particularly pertains to treating maladies of physically out-of-balance users according to the teachings of an ancient art, such treatment being for pain relief, reduction of inflammation, decreasing muscle spasms and reduction of edema.

2. Description of the Prior Art

The use of medical devices is known in the prior art. More specifically, medical devices previously devised and utilized for the purpose of treating various maladies are known to consist basically of familiar, expected, and obvious structural configurations, notwithstanding the myriad of designs encompassed by the crowded prior art which has been developed for the fulfillment of countless objectives and requirements.

While known devices fulfill their respective, particular objectives and requirements, the prior art does not describe a needle-less/wireless acupuncture system and method that allows treating maladies of physically out-of-balance users according to the teachings of an ancient art, such treatment being for pain relief, reduction of inflammation, decreasing muscle spasms and reduction of edema.

In this respect, the needle-less/wireless acupuncture system and method according to the present invention substantially departs from the conventional concepts and designs of the prior art, and in doing so provides an apparatus primarily developed for the purpose of treating maladies of physically out-of-balance users according to the teachings of an ancient art, such treatment being for pain relief, reduction of inflammation, decreasing muscle spasms and reduction of edema.

Therefore, it can be appreciated that there exists a continuing need for a new and improved needle-less/wireless acupuncture system and method which can be used for treating maladies of physically out-of-balance users according to the teachings of an ancient art, such treatment being for pain relief, reduction of inflammation, decreasing muscle spasms and reduction of edema. In this regard, the present invention substantially fulfills this need.

### SUMMARY OF THE INVENTION

In view of the disadvantages inherent in the known types of medical devices now present in the prior art, the present invention provides an improved needle-less/wireless acupuncture system and method. As such, the general purpose of the present invention, which will be described subsequently in greater detail, is to provide a new and improved needle-less/wireless acupuncture system and method which has all the advantages of the prior art and none of the disadvantages.

To attain this, the present invention essentially comprises a bio-electricity system and method including, but not limited to, electrical muscle stimulation, interferential current, micro-current, Russian stimulation, tens-transcutaneous electric nerve stimulator, and the like. The system and method are adapted for treating maladies of physically out-of-balance users according to the teachings of an ancient art. Such treatment is for pain relief, reduction of inflammation, decreasing muscle spasms and reduction of edema to other areas outside of the acupoint area. The system and method include a plurality of steps and components including probes, adhesive, electronic components, an instruction manual, an electronic device and a program in the electronic device to wirelessly activate probes positioned on a user in accordance with the instruction manual.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved needle-less/wireless acupuncture system and method which has all of the advantages of the prior art medical devices and none of the disadvantages.

It is another object of the present invention to provide a new and improved needle-less/wireless acupuncture system and method which may be easily and efficiently manufactured and marketed.

It is further object of the present invention to provide a new and improved needle-less/wireless acupuncture system and method which is of durable and reliable constructions.

An even further object of the present invention is to provide a new and improved needle-less/wireless acupuncture system and method which is susceptible of a low cost of manufacture with regard to both materials and labor, and which accordingly is then susceptible of low prices of sales, thereby making such needle-less/wireless acupuncture system and method economically available.

Even still another object of the present invention is to provide a needle-less/wireless acupuncture system and method for treating maladies of physically out-of-balance users according to the teachings of an ancient art, such treatment being for pain relief, reduction of inflammation, decreasing muscle spasms and reduction of edema.

Lastly, it is an object of the present invention to provide a new and improved system and method for treating a malady of a physically out-of-balance user according to the teachings of an ancient art, the treating adapted to stimulate proper body points of the user by create bioelectricity which thus stimulates conductance of nerve pathways for allowing the user to reach restored balance, homeostasis, the treating being done in a safe, convenient and economical manner.

These together with other objects of the invention, along with the various features of novelty which characterize the invention, are pointed out with particularity in the claims annexed to and forming a part of this disclosure.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a plan view of a sheet with probes, a portion of the needle-less/wireless acupuncture system constructed in accordance with the principles of the present invention.

FIG. 2 illustrates one probe including plan and side elevational views.

FIG. 3 is a front elevational view of a handheld electronic device with a holder therefor.

FIG. 4 is a perspective illustration of a hard copy of an instruction manual.

FIG. 5 is a plan view of a sheet with probes constructed in accordance with an alternate embodiment of the invention.

FIG. 6 illustrates one probe shown in FIG. 5 including plan and side elevational views.

FIG. 7 is a perspective illustration of a hard copy of an instruction manual for use with the alternate embodiment.

FIG. 8 is a front elevational view of a sales office for use with the alternate embodiment.

FIG. 9 is a front elevational view of a cell phone, the handheld electronic device for use with the alternate embodiment with a holder therefor.

The same reference numerals refer to the same parts throughout the various Figures.

### DESCRIPTION OF THE PREFERRED EMBODIMENT

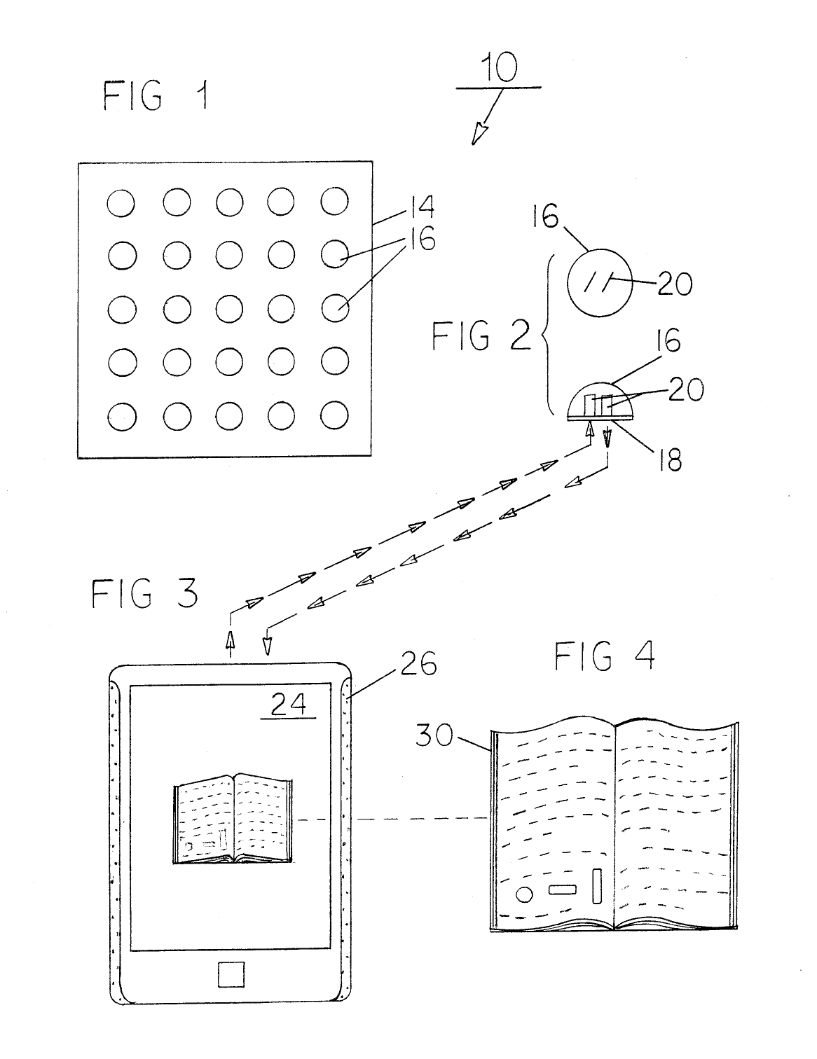

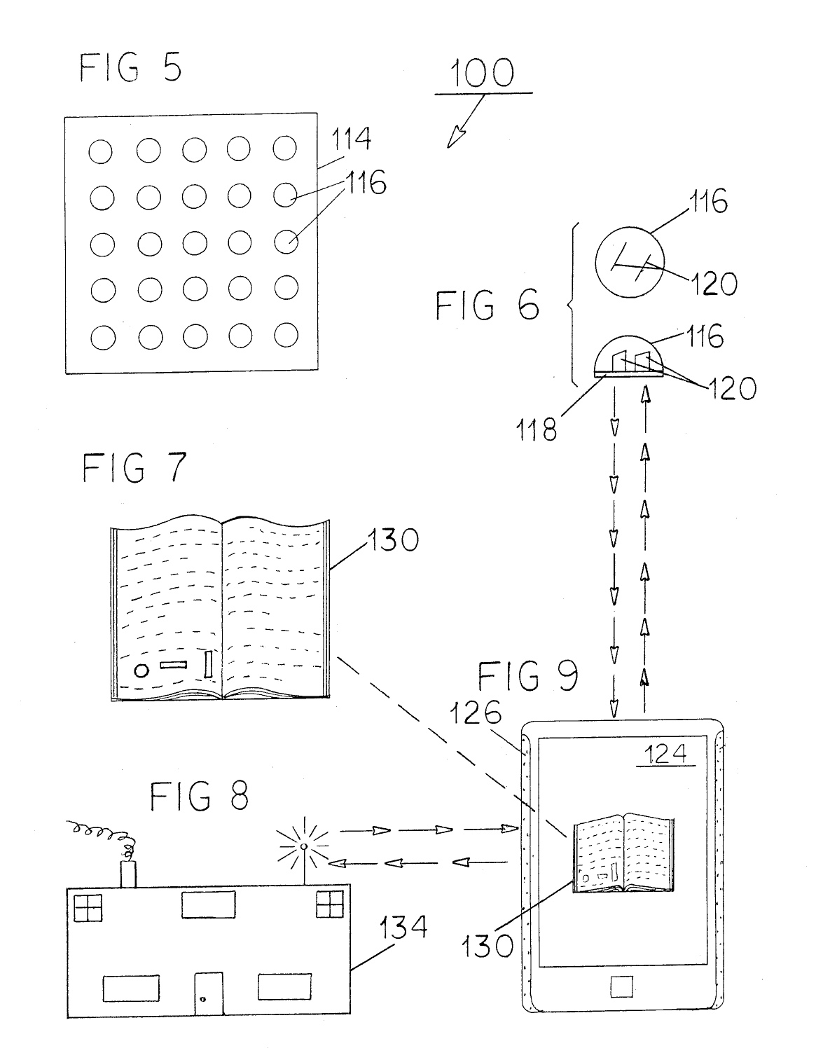

With reference now to the drawings, and in particular to FIGS. 1-4 thereof, the preferred embodiment of the new and improved needle-less/wireless acupuncture system and method embodying the principles and concepts of the present invention and generally designated by the reference numeral 10 will be described.

The present invention, the needle-less/wireless acupuncture system 10 and method is comprised of a plurality of components. Such components are individually configured and correlated with respect to each other so as to attain the desired objective. In their broadest context such components include a thin plastic sheet with a plurality of similarly configured probes, each probe having a layer of reusable adhesive and an electronic component, the components also including an instruction manual and an electronic device, the system also including a program in the electronic device to wirelessly activate probes positioned on a user in accordance with the instruction manual as a function of the malady to be treated.

The preferred embodiment of the needle-less/wireless acupuncture system, designated by reference numeral 10, includes a system for treating a malady of a physically out-of-balance user according to the teachings of an ancient art, the treating adapted to stimulate proper body points of the user by create bioelectricity which thus stimulates conductance of nerve pathways for allowing the user to reach restored balance, homeostasis.

The system comprises a thin plastic sheet 14, 114 formed in a rectangular configuration. A plurality of similarly configured probes 16, 116 are removably secured to the sheet in rows and columns. Each of the probes has a flat circular lower surface with a diameter. Each probe has a generally hemispherical upper surface with a height. The diameter is 0.50 inches plus or minus 10 percent. The height is between 0.25 inches and 0.125 inches. The diameter is between 2 and 4 times the height. A layer of reusable adhesive 18, 118 is between the lower surface of each probe and the sheet. A plurality of probes are adapted to be removed from the sheet and adhesively positioned on specific body points for acupuncture treatment and then adhesively re-secured to the sheet. An electronic component 20, 120 is imbedded within each probe. Each electronic component is adapted to generate an electric charge when activated. Each electronic component is adapted to receive a wireless activation signal and to activate the electronic components for a predetermined time in response to such activation signals.

Next provided is a handheld electronic device 24, 124. The handheld electronic device is adapted to function as a master in transmitting activation and inactivation signals to the electronic components functioning as slaves. The handheld electronic device is adapted to programmed by the user to transmit specific signals wirelessly as a function of the probes positioned on the user and the malady being to be treated. A carrying case 26, 126 is adapted to removably receive and support the handheld electronic device for keeping the handheld electronic device in close proximity to the user.

Provided last is an instruction manual 30, 130. The instruction manual is in hard copy form. The instruction manual is also in digital and downloadable form within the handheld device. The instruction manual has a listing of maladies to be treated, and for each listed malady, the number of probes to be utilized along with a body diagram identifying the body points for positioning the probes on the user.

In one embodiment of the invention, the handheld electronic device is a dedicated device 24.

In another embodiment of the invention, wherein the handheld electronic device is a cell phone 124.

In addition to the system as described above, the invention is also a needle-less/wireless acupuncture method for treating a malady of a physically out-of-balance user according to the teachings of an ancient art. The treating method is adapted to stimulate proper body points of the user by create bioelectricity which thus stimulates conductance of nerve pathways for allowing the user to reach restored balance, homeostasis.

The first step is providing a plurality of components as set forth above and including a thin plastic sheet 14 with a plurality of similarly configured probes 16. Each probe has a layer of reusable adhesive 18 and an electronic component 20. The plurality of components also include an instruction manual 30 and a handheld electronic device 24 with a carrying case 26.

The next step is installing onto the handheld electronic device the manual and the program for activating the probes wirelessly in response to the malady to be treated.

The final step is selling a kit. The kit includes the thin plastic sheet with the plurality of similarly configured probes, each probe having a layer of reusable adhesive and an electronic component. The kit also includes the handheld electronic device preprogrammed for activating the probes wirelessly in response to the malady to be treated. The kit also including the carrying case and the instruction manual.

In a final embodiment of the invention, the needle-less/wireless acupuncture method is for treating a malady of a physically out-of-balance user according to the teachings of an ancient art. The treating method is adapted to stimulate proper body points of the user by create bioelectricity which thus stimulates conductance of nerve pathways for allowing the user to reach restored balance, homeostasis.

The first step is providing a plurality of components as set forth above and including an instruction manual 130 and a thin plastic sheet 114. Each sheet has a plurality of similarly configured probes 116. Each probe has a layer of reusable adhesive 118 and an electronic component 120.

The next step is providing a sales office 134 to stock pluralities of components including instruction manuals 130 and thin plastic sheets 114, each having a plurality of similarly configured probes 116, each probe having a layer of reusable adhesive 118 and an electronic component 120.

The following step is utilizing a cell phone 124 to purchase components including an instruction manual 130 and a thin plastic sheet 114 having a plurality of similarly configured probes 116, each probe having an electronic component 120 and a layer of reusable adhesive 118. In this embodiment, the downloaded application preferably includes BLUETOOTH™ technology for operating the slave probes in response to the master handheld electronic device, a cell phone. The system is operable at locations with WI-FI technology and at locations without WI-FI technology. BLUETOOTH is a trademark of BLUETOOTH SIG, INC. of Kirkland, Wash. WI-FI is a trademark of WI-FI Alliance CORPORATION of Austin, Tex.

In all of the embodiments, the electronic device is programmed for low powered transmissions at open frequencies with a low energy wireless solution. The electronic device is preferably programmed with BLUETOOTH technology.

Next is the following step of downloading from the sales office to the cell phone an application. The application includes an operating program adapted to activate the probes wirelessly as a function of the malady being treated.

Last is the following step of forwarding the purchased components from the sales office to the user.

The present invention is adapted for utilizing electrical muscle stimulation, interferential current, micro-current, Russian stimulation and tens-transcutaneous electric nerve stimulator. The system is adapted for treating maladies of physically out-of-balance users according to the teachings of an ancient art. Such treatment is for pain relief, reduction of inflammation, decreasing muscle spasms and reduction of edema to other areas outside of the acupoint area. For example, active trigger points located in the upper back/lower neck region that require the wireless capability outside the traditional acupuncture protocol. This would allow the user to select the different types of current and areas to be treated not just limited to the acupoint system/acupuncture.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A bio-electricity system utilizing electrical muscle stimulation, interferential current, micro-current, Russian stimulation and tens-transcutaneous electric nerve stimulator, the system adapted for treating maladies of physically out-of-balance users according to the teachings of an ancient art, such treatment being for pain relief, reduction of inflammation, decreasing muscle spasms and reduction of edema to other areas outside of the acupoint area, the system including a plurality of components including a plurality of similarly configured probes, each probe having a layer of adhesive and an electronic component, the components also including an instruction manual and an electronic device, the system also including a program in the electronic device to wirelessly activate probes positioned on a user in accordance with the instruction manual as a function of the malady to be treated.

2. A needle-less/wireless acupuncture system 10 for treating a malady of a physically out-of-balance user according to the teachings of an ancient art, the treating adapted to stimulate proper body points of the user by create bioelectricity which thus stimulates conductance of nerve pathways for allowing the user to reach restored balance, homeostasis, the treating being done in safe, convenient and economical manner, the system comprising, in combination:
a thin plastic sheet 14 formed in a rectangular configuration, a plurality of similarly configured probes 16 removably secured to the sheet in rows and columns, each of the probes having a flat circular lower surface with a diameter, each probe having a generally hemispherical upper surface with a height, the diameter being 0.50 inches plus or minus 10 percent, the height being between 0.25 inches and 0.125 inches, the diameter being between 2 and 4 times the height, a layer of reusable adhesive 18 between the lower surface of each probe and the sheet, a plurality of probes adapted to be removed from the sheet and adhesively positioned on specific body points for acupuncture treatment and then adhesively re-secured to the sheet, an electronic component 20 imbedded within each probe, each electronic component adapted to generate an electric charge when activated, each electronic component adapted to receive a wireless activation signal and to activate the electronic components for a predetermined time in response to such activation signals;
a handheld electronic device 24, the handheld electronic device adapted to function as a master in transmitting activation and inactivation signals to the electronic components functioning as slaves, the handheld electronic device adapted to programmed by the user to transmit specific signals as a function of the probes positioned on the user and the malady being to be treated, a carrying case 26 adapted to removably receive and support the handheld electronic device for keeping the handheld electronic device in close proximity to the user; and
an instruction manual 30, the instruction manual being in hard copy form, the instruction manual also being in digital and downloadable form within the handheld device, the instruction manual having a listing of maladies to be treated and for each listed malady, the number of probes to be utilized and a body diagram identifying the body points for positioning the probes on the user.

3. The system as set forth in claim 2 wherein the handheld electronic device is a dedicated device.

4. The system as set forth in claim 2 wherein the handheld electronic device is a cell phone.

5. The system as set forth in claim 2 wherein the handheld electronic device is programmed for low powered transmissions at open frequencies with a low energy wireless solution.

6. The system as set forth in claim 5 wherein the handheld electronic device is programmed with BLUETOOTH technology.

7. A needle-less/wireless acupuncture method for treating a malady of a physically out-of-balance user according to the teachings of an ancient art, the treating method adapted to stimulate proper body points of the user by create bioelectricity which thus stimulates conductance of nerve pathways for allowing the user to reach restored balance, homeostasis, the treating being done in safe, convenient and economical manner, the method comprising, in combination, the steps of:
providing a plurality of components as set forth above including a thin plastic sheet with a plurality of similarly configured probes each with a layer of reusable adhesive and an electronic component, the plurality of components also including an instruction manual and a handheld electronic device with a carrying case;
installing onto the handheld electronic device the manual and the program for wirelessly activating the probes in response to the malady to be treated; and
selling a kit, the kit including the thin plastic sheet with the plurality of similarly configured probes, each probe having a layer of reusable adhesive and an electronic component, the kit also including the handheld electronic device preprogrammed for activating the probes wirelessly in response to the malady to be treated, the kit also including the carrying case and the instruction manual.

8. A needle-less/wireless acupuncture method for treating a malady of a physically out-of-balance user according to the teachings of an ancient art, the treating method adapted to stimulate proper body points of the user by create bioelectricity which thus stimulates conductance of nerve pathways for allowing the user to reach restored balance, homeostasis, the treating being done in safe, convenient and economical manner, the method comprising, in combination, the steps of:
providing a plurality of components as set forth above including an instruction manual and a thin plastic sheet having a plurality of similarly configured probes, each probe having a layer of reusable adhesive and an electronic component;
providing a sales office to stock pluralities of components including instruction manuals and thin plastic sheets each having a plurality of similarly configured probes, each probe having a layer of reusable adhesive and an electronic component;
utilizing a cell phone to purchase components including an instruction manual and a thin plastic sheet having a plurality of similarly configured probes, each probe having a layer of reusable adhesive and an electronic component;
forwarding the purchased components from the sales office to the user; and
downloading from the sales office to the cell phone an application including an operating program adapted to activate the probes wirelessly as a function of the malady being treated.
